(19) United States
(12) Patent Application Publication
(10) Pub. No.: US 2011/0208039 A1
(43) Pub. Date: Aug. 25, 2011

(54) Direct and Indirect Surface Coil Correction for Cardiac Perfusion MRI

(75) Inventors: Jens Guehring, Erlangen (DE); Hui Xue, Franklin Park, NJ (US); Sven Zuehlsdorff, Chicago, IL (US)

(73) Assignee: Siemens Corporation, Iselin, NJ (US)

(21) Appl. No.: 13/031,321

(22) Filed: Feb. 21, 2011

Related U.S. Application Data
(60) Provisional application No. 61/306,593, filed on Feb. 22, 2010.

(51) Int. Cl. A61B 5/055

(52) U.S. Cl. 600/410

(57) ABSTRACT

Method of correcting cardiac perfusion MR imaging for inhomogeneities (430) caused by non-uniform receiver coil fields using proton density weighted images (410) and B-Spline Free-Form Deformation (425).

### CROSS-REFERENCE TO RELATED APPLICATIONS

This application claims the benefit of Provisional U.S. Patent Application Ser. No. 61/306,593, entitled, “Direct and Indirect Surface Coil Correction for Cardiac Perfusion MRI”, filed in the name of Hui Xue, Jens Guehring, and Sven Zuehlsdorff, on Feb. 22, 2010, the disclosure of which is also hereby incorporated herein by reference.

### FIELD OF INVENTION

The present invention relates to cardiac imaging. More particularly, the present invention relates to cardiovascular magnetic resonance imaging to assess or measure myocardial blood flow.

### BACKGROUND OF THE INVENTION

Myocardial first pass perfusion magnetic resonance imaging (MRI) is a diagnostic imaging approach using cardiovascular magnetic resonance to assess or measure myocardial blood flow from the contrast enhancement observed during the first pass of a contrast agent bolus. It has proven its clinical significance in the diagnosis of known and suspected ischemic heart disease, particularly in combination with cardiac delayed enhancement imaging (this is reported in more detail in an article by P. Kellman and A. E. Ara, entitled “Imaging Sequences for First Pass Perfusion-A Review”, Journal of Cardiovascular Magnetic Resonance, Issue 10 (2007) pp 525-537). However, the clinical routine to evaluate myocardial perfusion MR images still relies on qualitative visual reading by a health practitioner which is subjective and suffers from inter-observer variability. Computing the myocardial perfusion quantitative maps from the pixel-wise or segmental perfusion signal intensity curves can bring in more objectivity. FIG. 1 shows a perfusion signal intensity curve and associated perfusion parameters. For each pixel of a myocardial perfusion MR image, the signal-time curve may be analyzed and the perfusion parameters may be calculated. The parameters include, for example, upslope (SLOPE), time-to-peak (TTP), peak time (PT) and area-under-curve between foot and peak (AUC, not specifically labeled). The graph notations t_{f} and tₚ are foot time and peak time, respectively. Typically, time may be measured in seconds and intensity in AU (arbitrary units).

Unfortunately, the B1-field inhomogeneity caused by non-uniform characteristics of the receiver coils employed in conventional MR imaging systems causes signal intensity variation which will affect quantitative assessment (see, for example, FIG. 2b) and must be corrected before computing the perfusion quantitative maps (this is further described in the Kellman and Ara article cited above). FIG. 2a shows a proton density (PD) image of a heart acquired before normal perfusion acquisition. A PD image is produced by controlling the selection of MR scan parameters to minimize the effects of T1 and T2, resulting in an image dependent primarily on the density of protons in the imaging volume. FIG. 2b shows an intensity profile (i.e., perfusion signal intensity) across the heart region 205 indicated in FIG. 2a. The intensity bias can be clearly observed in FIG. 2b. Although qualitative visual reading is often not compromised by the inhomogeneity (as reported in article by R. Guillemaud and M. Brady, entitled “Estimating bias field of MR images, IEEE Trans. Med. Imaging 16 (1997) pp. 238-251), it causes the drifting of signal intensities and leads to errors of quantitative perfusion analysis (as further described in the Kellman and Ara article cited above).

While MR cardiac perfusion imaging sequences and motion correction has been thoroughly investigated by many researchers (as reported in an article by M. B. Stegmann; H. Olafsdottir; and H. B. Larsson, entitled “Unsupervised motion-compensation of multi-slice cardiac perfusion MRI”, Med Image Anal. 9(4) (2005) pp 394-410), the B1-field inhomogeneity correction still lacks intensive studies. It is noted that inhomogeneity correction has been intensively studied in neuro-imaging and muscular skeletal-imaging. But, as indicated above, inhomogeneity correction has not acquired an equal amount of interest in the field of cardiac MR imaging. The few published solutions (for example, the solution described in an article by L. Hsu; K. L. Rhoads; A. H. Aletras, and A. E. Arai, entitled “Surface Coil Intensity Correction and Non-linear Intensity Normalization Improve Pixel-Resolution Parametric Maps of Myocardial MRI Perfusion”, MICCAI (2003) pp. 975-976) requires substantial manual delineating of the myocardium and applying global polynomial fitting on the image.

### SUMMARY OF THE INVENTION

The present invention obviates the aforementioned problems by providing a method of cardiac perfusion magnetic resonance (MR) imaging, comprising acquiring a proton density image of a target cardiac region using an MR imaging pulse sequence; acquiring an image series of the target cardiac region using an MR imaging pulse sequence; estimating intensity variations in the pixels of the proton density image; correcting the image series of the target cardiac region to compensate for the estimated intensity variations in the pixels of the proton density image; and generating a corrected image series of the target cardiac region. The proton density image and the image series may each be acquired using a part of the same MR imaging pulse sequence. Acquiring a proton density image may comprise excluding background pixels from the proton density image.

Further, the estimating step may comprise calculating an approximation of the B-Spline free-form deformation (FFD) from the proton density image data to generate an estimated bias field and the correcting step may comprise applying the estimated bias field to the image series of the target cardiac region. Alternatively, the estimating step may comprise calculating an approximation of the B-Spline free-form deformation (FFD) from the proton density image data to extract the low frequency component of the proton density image and the correcting step may comprise applying the low frequency image data to the image series of the target cardiac region.

The estimating step may also comprise registering the proton density image to the image series of the target cardiac region. In such case, the registering step may comprise averaging all registered proton density images to improve the signal-to-noise ratio. Alternatively, the estimating step may comprise compensating the proton density image for cardiac motion. In such case, the compensating step may comprise averaging all motion-compensated proton density images to improve the signal-to-noise ratio.

Further, the estimating step may comprise interleaving proton image tissue classification and intensity variation bias correction using an Expectation-Maximization (EM) algorithm and B-Spline free-form deformation (FFD) on the proton density image data to generate an estimated bias field and a background tissue map, and the correcting step may comprise applying the estimated bias field to the image series of the target cardiac region. In such case, the interleaving step may comprise performing tissue classification in the expectation step of the EM algorithm and updating the tissue classification estimation in the maximization step of the EM algorithm. As an alternative, the interleaving step may comprise calculating an approximation of the B-Spline free-form deformation (FFD) from the proton density image data to generate an estimated bias field, and optimizing the B-Spline FFD approximation in the maximization step of the EM algorithm. The estimating step may also comprise iteratively optimizing an approximation of the B-Spline free-form deformation (FFD) from the proton density image data to generate an estimated bias field, and the correcting step may comprise applying the estimated bias field to the image series of the target cardiac region.

The present invention also provides a method of magnetic resonance (MR) imaging, comprising estimating coil-induced intensity variations in a respective proton density image of a target anatomical region; and generating an image sequence of estimated intensity variation-compensated images of the target anatomical region. The proton density image and the image sequence may each be acquired using a part of the same MR imaging pulse sequence. The estimating step may comprise calculating an approximation of the B-Spline free-form deformation (FFD) from the proton density image data to generate an estimated bias field. In such case, the generating step may comprise applying the estimated bias field to the image sequence of the target anatomical region to compensate for the respective estimated intensity variations. Alternatively, the estimating step may comprise iteratively optimizing an approximation of the B-Spline free-form deformation (FFD) from the proton density image data to generate an estimated bias field, and the generating step may comprise applying the estimated bias field to the image sequence of the target anatomical region to compensate for the respective estimated intensity variations.

The present invention also provides a method of evaluating magnetic resonance (MR) images, comprising estimating receiver coil-induced intensity variations in a respective proton density image of a target anatomical region; generating a series of estimated intensity variation-compensated images of the target anatomical region; and generating a quantitative map from pixel-wise or segmental signal intensity curves of the estimated intensity variation-compensated image series. The estimating step may comprise calculating an approximation of the B-Spline free-form deformation (FFD) from the proton density image data to generate an estimated bias field. In such case, the generating a series step may comprise applying the estimated bias field to the image series of the target anatomical region to compensate for the respective estimated intensity variations. The generating a quantitative map step may comprise calculating and presenting map parameters related to characteristics of the target anatomical region. Alternatively, the estimating step may comprise iteratively optimizing an approximation of the B-Spline free-form deformation (FFD) from the proton density image data to generate an estimated bias field, and the generating a series step may comprise applying the estimated bias field to the image series of the target anatomical region to compensate for the respective estimated intensity variations.

### DESCRIPTION OF THE DRAWINGS

For a better understanding of the present invention, reference is made to the following description of an exemplary embodiment thereof, and to the accompanying drawings, wherein:

FIG. 1 shows a perfusion signal intensity curve and associated perfusion parameters;

FIG. 2a shows a proton density cardiac image acquired before normal perfusion acquisition;

FIG. 2b shows an intensity profile across a heart region of FIG. 2a;

FIG. 3 is an MR imaging system (simplified) that performs MR imaging in accordance with the present invention;

FIG. 4 is a flow chart of a method of MR imaging carried out in accordance with the present invention;

FIG. 5 is a flow chart of an alternative method of MR imaging carried out in accordance with the present invention;

FIG. 6a shows the estimated bias field for the PD image in FIG. 2a using the method of MR imaging of FIG. 5;

FIG. 6b shows the corrected intensity profile for the PD image in FIG. 2a using the method of MR imaging of FIG. 5;

FIG. 7 is an illustration of the iterative scheme of the alternative method of MR imaging of FIG. 5;

FIG. 8a is a first frame of a GRE-EPI perfusion image series from a validation experiment of a method of the present invention;

FIG. 8b is the intensity profile across a heart region of the first frame image of FIG. 8a showing clear inhomogeneity;

FIG. 8c is the corrected first frame for the perfusion image of FIG. 8a using an indirect SCC; and

FIG. 8d is the improved intensity profile for the perfusion image of FIG. 8a.

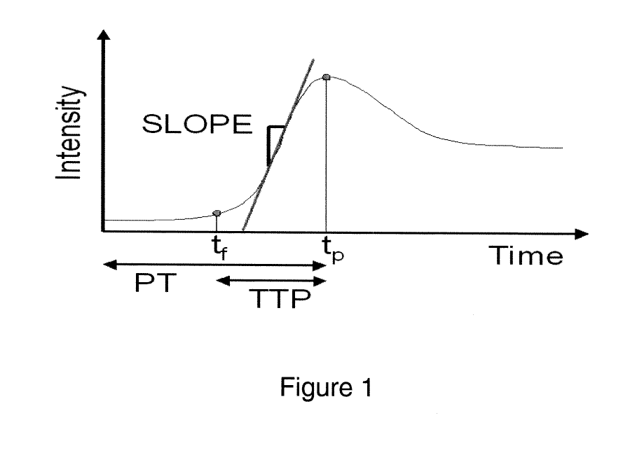

### DETAILED DESCRIPTION

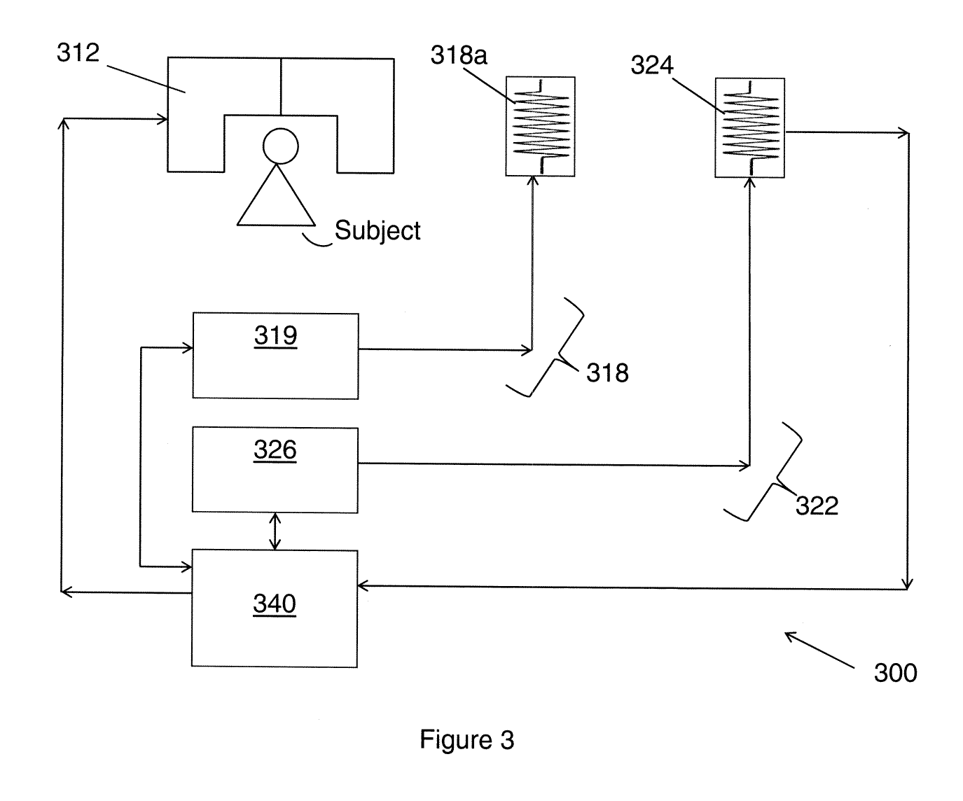

FIG. 3 is a block diagram of a conventional MRI scanner 300 (simplified) that performs cardiac perfusion MR imaging in accordance with the present invention. A main magnet 312 generates a strong static magnetic field in an imaging region where the subject (i.e., patient) is introduced. The magnet 312 is used to polarize the target cardiac area, i.e., certain atoms in the target cardiac area that were previously randomly-ordered become aligned along the magnetic field. A gradient coil system 318, having a gradient coil subsystem 318a and a gradient coil control unit 319, generates a time-varying linear magnetic field gradient in respective spatial directions, x, y and z, and spatially encodes the positions of the polarized or excited atoms. An RF system 322, having an RF coil subsystem 324 and a pulse generation unit 326, transmits a series of RF pulses to the target cardiac region to excite the “ordered” atoms of the target cardiac area. The RF coil subsystem 324 may be adapted to switch between a transmission mode and receiver mode.

A control or computer system 340 coordinates the pulse generation unit 326, the gradient coil control unit 319, and other components to carry out a desired MR image pulse sequence. The scanner 300 repeats the MR image pulse sequence a number of times so the atoms oscillate around the polarized alignment direction (along the main magnetic field) during the excited state caused by the energy of RF pulses. The atoms release the RF energy, i.e., generate an RF signal, during the resonance or oscillation and as the atoms return to their respective alignments. The RF coil subsystem 324 receives or detects the released RF energy and generates spatially-coded MR signals to the computer system 340. It is noted that the subject may be injected with contrast agent that permeates the target cardiac area in order to assist in the capture of image data and the resulting image visualization.

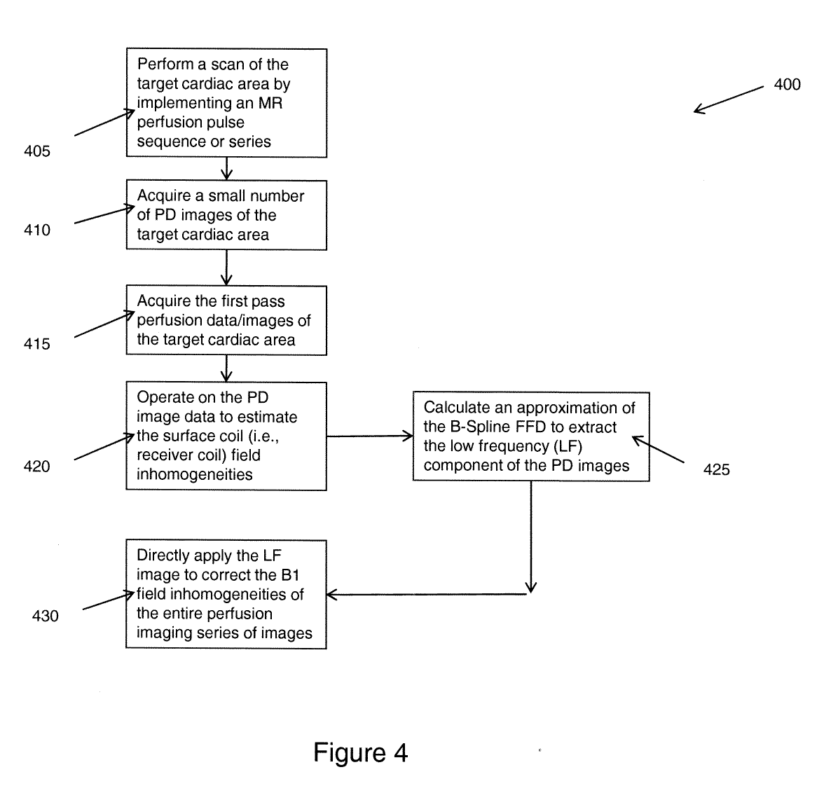

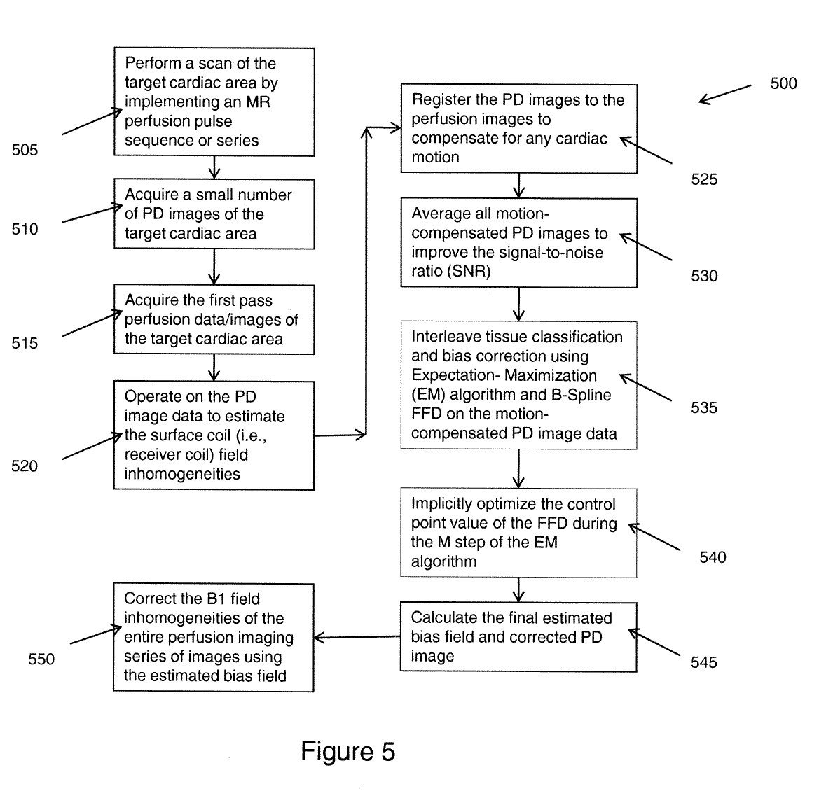

The computer system 340, which controls the operation of the MR scanner 300 and its components, processes the MR signals to transform them into a visual representation of the target cardiac region (i.e., reconstructed MR images) for display, storage, and/or other usage. As noted above, non-uniform characteristics of the receiver RF coils cause B1-field inhomogeneities which then cause variations in the signal intensity profiles for the MR images. This will affect quantitative assessments of the MR images. To correct or compensate for these field inhomogeneities, the MR scanner 300 and, in particular, the computer system 340, is adapted to permit the imaging scanner 300 to operate and to implement methods of the present invention, for example, as shown in FIGS. 4 and 5.

The present invention provides methods to overcome the inhomogeneity caused by the non-uniform receiver coils via approaches to perform surface coil (i.e., receiver coil) inhomogeneity correction (SCC) using PD weighted images and B-Spline Free-Form Deformation (FFD). Generally, B-Spline FFD is a technique for reconstructing an image (or graphing/fitting a surface) from scattered, or nonuniform, distribution of data samples (this is more fully described in an article by S. Y. Lee, G. Wolberg, and S. Y. Shin, entitled “Scattered data interpolation with Multilevel BSplines”, IEEE Trans Visualization and Computer Graphics Vol. 3(3), 1997, pp. 228-244). These approaches rely on the acquisition of PD images before the perfusion read-out to estimate the receiver coil-introduced intensity variation within the Field-of-View (FOV) of the cardiac perfusion imaging. It is assumed that the intensity variation in the PD images is largely driven by the field inhomogeneity of the receiver coils.

FIG. 4 shows a flow chart of a first method of performing MR imaging 400 carried out in accordance with the present invention. Generally, the first method 400 directly approximates the field inhomogeneity. A health practitioner operates the MR imaging system 300 to perform a scan of the target cardiac area by implementing an MR perfusion pulse sequence or series (Step 405). The pulse sequence is designed to carry out myocardial first pass perfusion MR imaging. The pulse sequence is also designed to acquire a small number of PD images of the target cardiac area prior to the first pass perfusion data/image acquisition. Accordingly, the MR scanner 300 acquires a small number of PD images of the target cardiac area (Step 410). The MR scanner 300 also acquires the first pass perfusion data/images of the target cardiac area (Step 415). The computer system 340 operates on the PD image data to estimate the surface coil field inhomogeneities (Step 420).

The method 400 assumes that the proton density across the myocardial anatomy is constant (as described in the Kellman and Ara article cited above) and, as mentioned above, that the signal intensity profile changes of the obtained PD images are positively related to local surface coil sensitivity. Accordingly, the computer system 340 calculates an approximation of the B-Spline FFD to extract the low frequency (LF) component of the PD images (Step 425). This LF image is then directly applied to correct the B1 field inhomogeneity of the entire perfusion imaging series of images (Step 430). To eliminate the influences of background pixels, the Otsu method may be performed iteratively to find an optimal threshold (this is described in an article by Nobuyuki Otsu, entitled “A threshold selection method from gray-level histograms”, IEEE Trans. Sys., Man., Cyber., Vol. 9, 1979, pp 62-66).

The B-Spline FFD applied to approximate the bias field (to correct or compensate for the field inhomogeneity) parameterizes a dense 2D bias field at a sparse control point lattice. One may define the field-of-view (FOV) of the PD image as follows: Ω_{S}={(x,y)/0≦x≦X, 0≦y≦Y} and Φ_{S} denotes a grid of control points φ_{p,q} with the grid spacing being Δₓ×Δ_{y}. This spacing between adjacent control points is uniform for each coordinate direction. The 2D tensor of uniform 1D cubic B-splines is used to represent the spatial-variant bias ratio \tilde{b}ᵢ:

$${T}_{local} ( {\tilde{b}}_{i} ) = {\sum }_{m = 0}^{3} {\sum }_{n = 0}^{3} {B}_{m} ( u ) {B}_{n} ( v ) {ϕ}_{p + m , q + n} , \qquad ( 1 )$$

where (x, y) is the coordinate of pixel i, and p=└x/Δₓ┘−1, q==└y/Δ_{y}┘−1, u=x/Δₓ−└x/Δₓ┘, and v=y/Δ_{y}−└y/Δ_{y}┘.
Bₘ represents the m-th basis function of the B-spline. The basis functions of cubic B-splines have limited support. Therefore, changing a control point in the grid affects only a 4×4 region around that control point.

FIG. 5 shows a flow chart of a second method of performing MR imaging 500 carried out in accordance with the present invention. Unlike the direct approximation approach of the first method 400, the second method 500 indirectly approximates the field inhomogeneity by interleaving the PD image tissue classification and bias correction, using the Expectation-Maximization (EM) algorithm, and the B-Spline FFD. The Expectation-Maximization (EM) algorithm generally is a method for finding maximum likelihood estimates of unknown parameters, given measurement data. It is an iterative method which alternates between performing an expectation (E) step in which missing data are estimated given the observed data and current estimate of the model parameters and the maximization (M) step in which the likelihood function is maximized under the assumption that the missing data are known. The estimate of the missing data from the E-step are used in lieu of the actual missing data (described in further detail in an article by A. P. Dempster, N. M. Laird, D. B. Rubin, entitled “Maximum likelihood from incomplete data via the EM algorithm”, Journal of the Royal Statistical Society, Vol. 39, 1977, pp. 1-38).

A health practitioner operates the MR imaging system 300 to perform a scan of the target cardiac area by implementing an MR perfusion pulse sequence or series (Step 505). The pulse sequence is designed to carry out myocardial first pass perfusion MR imaging. The pulse sequence is also designed to acquire a small number of PD images of the target cardiac area prior to the first pass perfusion data/image acquisition. Accordingly, the MR scanner 300 acquires a small number of PD images of the target cardiac area (Step 510). The MR scanner 300 also acquires the first pass perfusion data/images of the target cardiac area (Step 515). The computer system 340 operates on the PD image data to estimate the surface coil field inhomogeneities (Step 520).

The computer system 340 first registers the PD images to the perfusion images (i.e., transforms the different sets of data into one coordinate system) to compensate for any cardiac motion (Step 525). All motion-compensated PD images are then averaged to improve the signal-to-noise ratio (SNR) (Step 530).

As noted above, the computer system 340 interleaves the PD image tissue classification and bias correction using the Expectation-Maximization (EM) algorithm and the B-Spline FFD on the motion-compensated PD image data (Step 535). The EM algorithm consists of an expectation step (E-step) which performs tissue classification and a maximization step (M-step) which updates the parameter estimation. Assuming a Gaussian distribution and given initial parameters, the EM algorithm iteratively maximizes the data likelihood and updates the tissue classification. The present invention classifies PD images into three classes: background (BG), tissue with low intensity (TL) and tissue with high intensity (TH). This classification scheme is sufficient for the purposes of PD image-based bias correction because the contrast level in PD images is not sufficient to delineate specific tissue classes and the objective is not to obtain a detailed segmentation. The classification scheme may be different, however, for other applications of the present invention. Based on experimental results (described below), the three-class assumption is robust for separating the regions of background and lung from organ tissues. To improve the accuracy of inhomogeneity estimation, the computer system 340 may exclude all background pixels from further computations using any appropriate method.

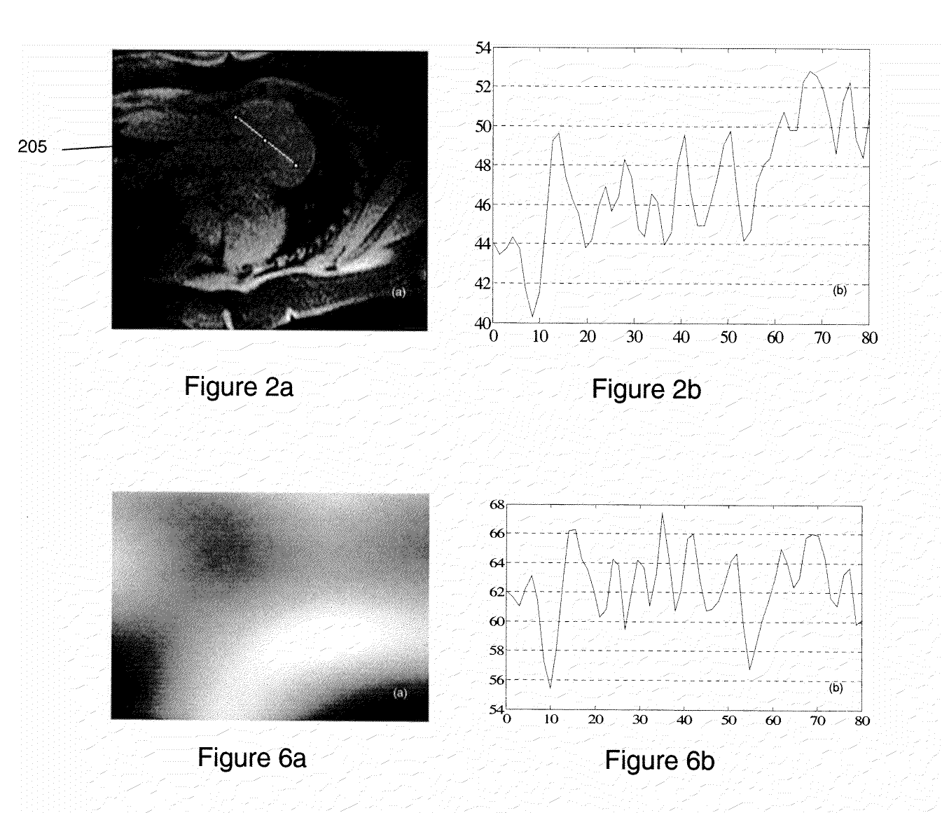

For the bias correction, the second method 500 assumes a multiplicative bias field. For a pixel i, its measured intensity is xᵢ, Defining the bias field at location i as bᵢ, the unbiased signal rᵢ can be estimated by xᵢ=rᵢ·bᵢ. Using the notation \tilde{x}ᵢ≦log(xᵢ), the image formation model can become additive \tilde{x}ᵢ=\tilde{r}ᵢ+\tilde{b}ᵢ. Then, corresponding mean and sigma become \tilde{μ}ₖ and \tilde{σ}ₖ. The second method 500 does not explicitly optimize the control point value during the M-step, because it leads to solving a linear system for every pixel in the image due to the local support of B-Spline. To find the optimal control point value, the present method 500 estimates a ‘bias-free’ image:

$${\tilde{r}}_{i}^{( m )} = \frac{{\sum }_{i = 1}^{3} {p}^{( m )} ( k \vert {x}_{i} ) \cdot {\tilde{μ}}_{k}^{( m )}}{{\sum }_{i = 1}^{3} {p}^{( m )} ( k \vert {x}_{i} )} , \qquad ( 2 )$$

where \tilde{r}ᵢ^{(m)} denotes the estimated real signal at pixel location i for iteration m. Then, the bias for this iteration can be estimated as \tilde{b}ᵢ^{(m)}=approx(\tilde{x}ᵢ^{(m)}−\tilde{r}ᵢ^{(m)}). The term approx( ) is the FFD approximation step, which calculates the optimal control point value. Given the estimated bias field, the corrected signal can be updated as \tilde{x}ᵢ^{(m+1)}=\tilde{x}ᵢ^{(m)}−\tilde{b}ᵢ^{(m)}. Thus, the second method 500 implicitly optimizes the control point value of FFD during the M-step (Step 540). Once the iteration converges or a maximum number of iterations is reached, the final estimated bias field and corrected PD image are calculated by an exponential operator (Step 545). As an illustration, FIG. 6 shows the estimated multiplicative bias field (FIG. 6a) and corrected intensity profile (FIG. 6b) for the PD image in FIG. 2a. The estimated bias field is used to correct the entire perfusion time series (Step 550).

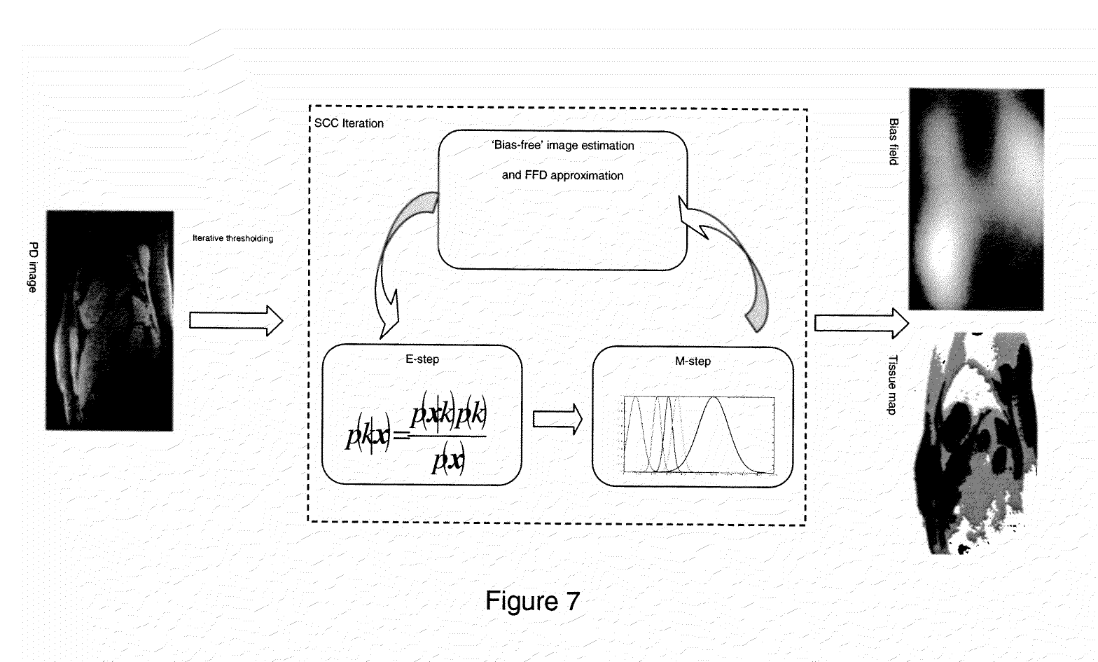

The iterative scheme of the second method 500 is illustrated in FIG. 7. The interleaving of tissue classification and bias correction using an EM approach is essentially an optimization step, introducing more accuracy and providing a computation of an optimal bias field and background tissue mask.

The feasibility of the methods 400, 500 of the present invention was verified on actual patient datasets and the experimental results are described as follows: To acquire PD images along with the conventional perfusion images, a MR perfusion pulse sequence was implemented and tested on two clinical 1.5 T scanners (specifically, MAGNETOM Avanto and MAGNETOM Espree by Siemens). The pulse sequence supports the commonly used readout modules, TurboFLASH, TrueFISP, and GRE-EPI, respectively. The pulse sequence was modified to first acquire a small number (e.g., two) of PD images prior to the start of the first pass perfusion data acquisition.

Validation was performed on anonymized data from 40 subjects, with a total of 260 perfusion series. Three different MR perfusion imaging sequences (74 of TurboFLASH, 12 of TrueFISP, and 174 of GRE-EPI) were used in these scans. All scans were performed with a minimum of three slice positions (basal, mid-ventricular and apical) and 2 PD images were acquired before the perfusion acquisition. The inhomogeneity correction fields estimated from the PD images were applied to the entire perfusion series to correct for the bias introduced by the surface coils.

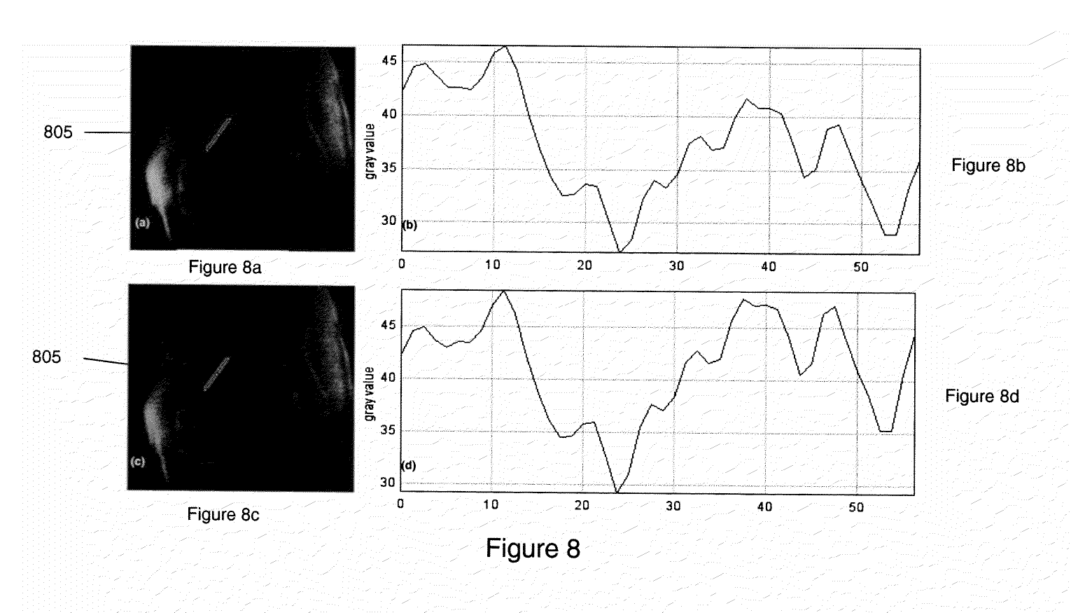

Visual inspection showed the reduction of intensity inhomogeneity that was consistently discernible throughout the whole data cohort. To quantitatively verify the effects of bias correction, the first frame of the perfusion acquisition was selected and the intensity profile across the heart was measured. A straight line was fitted to the data and the absolute slope (AS) with and without bias correction was measured. Because saturation recovery or inversion recovery pulses are normally applied to null the pre-contrast blood and tissue in the perfusion imaging, the intensity profile of the first phase often shows bias through the heart. For a group of 20 randomly selected series from the whole data cohort, the mean AS was originally 0.17±0.13 and reduced to 0.06±0.07 for indirect SCC and 0.08±0.04 for direct SCC. An illustration of indirect SCC performance is provided in FIG. 8. More specifically, FIG. 8a shows a first frame of a GRE-EPI perfusion image series from a validation experiment. FIG. 8b is the intensity profile across a heart region 805 of the first frame image of FIG. 8a showing clear inhomogeneity. FIG. 8c is the corrected first frame for the perfusion image of FIG. 8a using an indirect SCC. FIG. 8d is the improved intensity profile for the perfusion image of FIG. 8a from using an indirect SCC.

As a conclusion, both SCC approaches count on the B-Spline FFD to estimate the bias field. While the first method 400 (direct method) is more computationally efficient, the second method 500 (indirect method) shows higher accuracy.

The methods of the present invention are fully automated and no manual interaction is required during the operation of the MR scanner 300, which suits itself well for the scenario of unsupervised quantitative perfusion analysis. Compared to a simple polynomial fitting strategy, the FFD has the advantage of limited supports; that is, the value of a control point is only determined by its neighboring pixel values, while the smoothness of the estimated bias field is well maintained by the B-Spline. This feature leads to more accurate approximation of potential intensity variation. Furthermore, as noted above, in the indirect approximation approach, the interleaving of tissue classification and bias correction using an EM scheme is essentially an optimization step, introducing more accuracy and offering chances to computing an optimal bias field and background tissue mask. As a result, the methods of the present invention offer advantages compared to the simple polynomial fitting which has no guarantee to be optimal,

Each of the methods 400, 500 of the present invention have potential applicability beyond perfusion imaging, as other imaging sequences can be easily modified to combine a similar PD acquisition. The methods may have particular usefulness in applications where the intensity inhomogeneity may jeopardize qualitative/quantitative image assessment,

Other modifications are possible within the scope of the invention. For example, the subject patient to be scanned may be an animal subject or any other suitable object instead of a human patient. Also, the various components of the imaging scanner 300 are conventional and well known components. They may be configured and interconnected in various ways as necessary or as desired.

Further, although the steps of each method have been described in a specific sequence, the order of the steps may be re-ordered in part or in whole. Further, although in the described methods the health professional may use self-contained imaging instrumentation and tools, the health professional may use other instrumentation or tools in combination with or in place of the imaging instrumentation and tools described for any step or all the steps of the methods, including those that may be made available via telecommunication means. Further, the described methods, or any of their steps, may be carried out automatically by appropriate imaging instrumentation and tools or with some or minimal manual intervention.

What is claimed is:

1. A method of cardiac perfusion magnetic resonance (MR) imaging, comprising:
a. acquiring a proton density image of a target cardiac region using an MR imaging pulse sequence;
b. acquiring an image series of the target cardiac region using an MR imaging pulse sequence;
c. estimating intensity variations in the pixels of the proton density image;
d. correcting the image series of the target cardiac region to compensate for the estimated intensity variations in the pixels of the proton density image; and
e. generating a corrected image series of the target cardiac region.

2. The method of claim 1, wherein the proton density image and the image series are each acquired using a part of the same MR imaging pulse sequence.

3. The method of claim 1, wherein acquiring a proton density image comprises excluding background pixels from the proton density image.

4. The method of claim 1, wherein the estimating step comprises calculating an approximation of the B-Spline free-form deformation (FFD) from the proton density image data to generate an estimated bias field and the correcting step comprises applying the estimated bias field to the image series of the target cardiac region.

5. The method of claim 1, wherein the estimating step comprises calculating an approximation of the B-Spline free-form deformation (FFD) from the proton density image data to extract the low frequency component of the proton density image and the correcting step comprises applying the low frequency image data to the image series of the target cardiac region.

6. The method of claim 1, wherein the estimating step comprises registering the proton density image to the image series of the target cardiac region.

7. The method of claim 1, wherein the estimating step comprises compensating the proton density image for cardiac motion.

8. The method of claim 6, wherein the registering step comprises averaging all registered proton density images to improve the signal-to-noise ratio.

9. The method of claim 7, wherein the compensating step comprises averaging all motion-compensated proton density images to improve the signal-to-noise ratio.

10. The method of claim 1, wherein the estimating step comprises interleaving proton image tissue classification and intensity variation bias correction using an Expectation-Maximization (EM) algorithm and B-Spline free-form deformation (FFD) on the proton density image data to generate an estimated bias field and a background tissue map, and the correcting step comprises applying the estimated bias field to the image series of the target cardiac region.

11. The method of claim 10, wherein the interleaving step comprises performing tissue classification in the expectation step of the EM algorithm and updating the tissue classification estimation in the maximization step of the EM algorithm.

12. The method of claim 10, wherein the interleaving step comprises calculating an approximation of the B-Spline free-form deformation (FFD) from the proton density image data to generate an estimated bias field, and optimizing the B-Spline FFD approximation in the maximization step of the EM algorithm.

13. The method of claim 1, wherein the estimating step comprises iteratively optimizing an approximation of the B-Spline free-form deformation (FFD) from the proton density image data to generate an estimated bias field, and the correcting step comprises applying the estimated bias field to the image series of the target cardiac region.

14. A method of magnetic resonance (MR) imaging, comprising:
a. estimating coil-induced intensity variations in a respective proton density image of a target anatomical region; and
b. generating an image sequence of estimated intensity variation-compensated images of the target anatomical region.

15. The method of claim 14, wherein the proton density image and the image sequence are each acquired using a part of the same MR imaging pulse sequence.

16. The method of claim 14, wherein the estimating step comprises calculating an approximation of the B-Spline free-form deformation (FFD) from the proton density image data to generate an estimated bias field.

17. The method of claim 16, wherein the generating step comprises applying the estimated bias field to the image sequence of the target anatomical region to compensate for the respective estimated intensity variations.

18. The method of claim 14, wherein the estimating step comprises iteratively optimizing an approximation of the B-Spline free-form deformation (FFD) from the proton density image data to generate an estimated bias field, and the generating step comprises applying the estimated bias field to the image sequence of the target anatomical region to compensate for the respective estimated intensity variations.

19. A method of evaluating magnetic resonance (MR) images, comprising:
a. estimating receiver coil-induced intensity variations in a respective proton density image of a target anatomical region;
b. generating a series of estimated intensity variation-compensated images of the target anatomical region; and
c. generating a quantitative map from pixel-wise or segmental signal intensity curves of the estimated intensity variation-compensated image series.

20. The method of claim 19, wherein the estimating step comprises calculating an approximation of the B-Spline free-form deformation (FFD) from the proton density image data to generate an estimated bias field.

21. The method of claim 20, wherein the generating a series step comprises applying the estimated bias field to the image series of the target anatomical region to compensate for the respective estimated intensity variations.

22. The method of claim 19, wherein the generating a quantitative map step comprises calculating and presenting map parameters related to characteristics of the target anatomical region.

23. The method of claim 19, wherein the estimating step comprises iteratively optimizing an approximation of the B-Spline free-form deformation (FFD) from the proton density image data to generate an estimated bias field, and the generating a series step comprises applying the estimated bias field to the image series of the target anatomical region to compensate for the respective estimated intensity variations.
